(19) United States
(12) Patent Application Publication
(10) Pub. No.: US 2009/0036891 A1
(43) Pub. Date: Feb. 5, 2009

(54) ORTHOPAEDIC FIXATION CLAMP AND METHOD

(75) Inventors: Daniel G. Brown, Goshe, IN (US); Keith Denlinger, Warsaw, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER TECHNOLOGY, INC., Warsaw, IN (US)

(21) Appl. No.: 12/245,167

(22) Filed: Oct. 3, 2008

Related U.S. Application Data
(63) Continuation of application No. 11/201,021, filed on Aug. 9, 2005, now abandoned.

(51) Int. Cl. A61B 17/00

(52) U.S. Cl. 606/57; 606/54

(57) ABSTRACT

An orthopaedic fixation clamp for use in an external fixation system and its method of use are presented.

### CROSS-REFERENCE TO RELATED APPLICATIONS

This continuing patent application claims priority under 35 U.S.C. §120 to co-pending nonprovisional U.S. patent application Ser. No. 11/201,021, entitled ORTHOPAEDIC FIXATION CLAMP AND METHOD, filed on Aug. 9, 2005, the entire disclosure of which is expressly incorporated by reference herein.

### FIELD OF THE INVENTION

The present invention relates to external fixation devices for supporting bones in a desired position relative to one another. In particular, the present invention relates to clamps for use with pins and rods in an external fixation system.

### BACKGROUND

External fixation devices provide support to bones to treat fractures, immobilize joints, and otherwise position bones in orthopaedic procedures. In a typical external fixation device, pins are embedded into bones, or bone fragments, that are to be positioned relative to one another. The pins are connected to support rods by clamps that grip the pins and rods and lock them together in a desired position. Various combinations of clamps, rods, and pins may be assembled to fit a particular surgical geometry. External fixation may be used as the primary means for managing a bone fracture. External fixation may also be used as a temporary way to support soft tissues and maintain fracture alignment until another procedure such as periarticular plating or IM nailing can be performed.

FIG. 1 illustrates a prior art clamp assembly 10 manufactured by Zimmer, Inc., and sold under the trade name TransFx™. The TransFx™ clamp assembly 10 includes a shaft 12 having first and second threaded ends 14, 16. A rod clamp 18 includes a transverse bore (not shown) engaged with the shaft 12. The rod clamp 18 includes a first leg 20 fixed to the shaft 12 with a transverse pin 22 through the leg 20 and shaft 12. The rod clamp includes a second leg 24 connected to the first leg 20 to define a rod receiving opening 26. A pin clamp 28 includes a transverse bore (not shown) engaged with the shaft 12. The pin clamp 28 includes first and second halves 30, 32 defining a pin receiving opening 34. The pin clamp 28 abuts the rod clamp 18 and both the pin clamp 28 and the rod clamp 18 include serrations 36 to allow them to be locked together in a plurality of discrete angular positions relative to one another. First and second nuts 38, 40 thread onto the first and second threaded ends 14, 16 to tighten the clamps 18, 28. Tightening the first nut 38 against the pin clamp 28 presses the second half 32 of the clamp toward the first half 30 to grip a pin within the pin receiving opening 34. Tightening the first nut 38 also presses the serrations 36 together to lock the relative angular position of the pin and rod clamps 28, 18. Tightening the second nut 40 against the rod clamp 18 presses the second leg 24 of the rod clamp 18 toward the first leg 20 to grip a rod within the rod receiving opening 26. The rod clamp 18 is constrained by the transverse pin 22 to allow independent tightening of the pin and rod clamps 28, 18. However, once the pin clamp 28 is tightened, the relative angular position of the pin and rod clamps 28, 18 is fixed even while the rod clamp 18 remains loose such that the gripping action of the pin clamp 28 and angular positioning of the pin and rod clamps 28, 18 cannot be independently adjusted.

FIG. 2 illustrates a prior art clamp assembly 50 manufactured by Synthes. The assembly 50 includes a shaft 52 having a first threaded end 54 and a second end 55 defining an enlarged head 56. A first clamp 58 includes first and second halves 60, 62 defining a rod receiving opening 64 and a pin receiving opening 66. The first clamp includes a transverse bore (not shown) engaged with the shaft 52 so that the first half 60 abuts the enlarged head 56. A second, identical, clamp 68 includes first and second halves 70, 72 defining a rod receiving opening 74 and a pin receiving opening 76. The second clamp 68 includes a transverse bore (not shown) engaged with the shaft 52 so that the first half 70 abuts the first clamp 58. Serrations 79 formed on the clamps 58, 68 allow them to be locked together in a plurality of discrete relative angular positions. A nut 78 engages the threaded end 54 of the shaft and is tightened to compress the clamp halves together to grip pins and rods and to simultaneously lock the relative angular position of the clamps. The clamps are constrained on the shaft 52 by the enlarged head 56. The rod and pin gripping action of the clamps and the angular positioning of the clamps are simultaneously controlled by the nut 78 such that clamping and angular positioning cannot be independently adjusted.

### SUMMARY

The present invention provides an orthopaedic fixation clamp for use in an external fixation system and a method of using the clamp.

In one aspect of the invention, an orthopaedic fixation clamp for gripping first and second elongated members includes an elongated clamp shaft having a first end, a second end, and a longitudinal shaft axis extending therebetween. A first clamp body is mounted to the clamp shaft for rotation about the shaft axis and includes a first clamp opening able to receive the first elongated member. A second clamp body is mounted to the clamp shaft for rotation about the shaft axis and includes a second clamp opening able to receive the second elongated member. The first and second clamp bodies may be independently tightened to grip the elongate members and the rotational position of the clamp bodies may be independently locked relative to the shaft.

In another aspect of the invention, an orthopaedic fixation clamp assembly includes an elongated bone fixation member, an elongated interconnecting rod, and a clamp. The clamp includes an elongated clamp shaft having a first end, a second end, and a longitudinal shaft axis extending therebetween. A plurality of clamp bodies is mounted to the shaft. Each body includes a through bore engaged with the shaft for translation along the shaft axis and each body includes two opposing halves defining a hinge between them on one side of the shaft axis. Each body defines a relatively smaller fixation member receiving opening and a relatively larger rod receiving opening with each of the opposing halves defining one half of the fixation member receiving opening and one half of the rod receiving opening. The opposing halves are hingeable toward one another to reduce both openings and hingeable away from one another to enlarge both openings. Both the fixation member receiving opening and the rod receiving opening are formed in each of the clamp bodies transverse to the shaft axis and opposite the hinge with the shaft between the openings and the hinge.

In another aspect of the invention, a method of fixing a first bone portion relative to a second bone portion includes inserting fixation members into the bone portions and connecting the fixation members to a connecting rod with clamps.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a side elevation view of a prior art fixation clamp;

FIG. 2 is a side elevation view of a prior art fixation clamp;

FIG. 3 is an exploded perspective view of an exemplary clamp assembly according to the present invention;

FIG. 4 is a side elevation view of the clamp assembly of FIG. 3; and

FIG. 5 is a perspective view of the clamp assembly of FIG. 3 in use in an orthopaedic procedure.

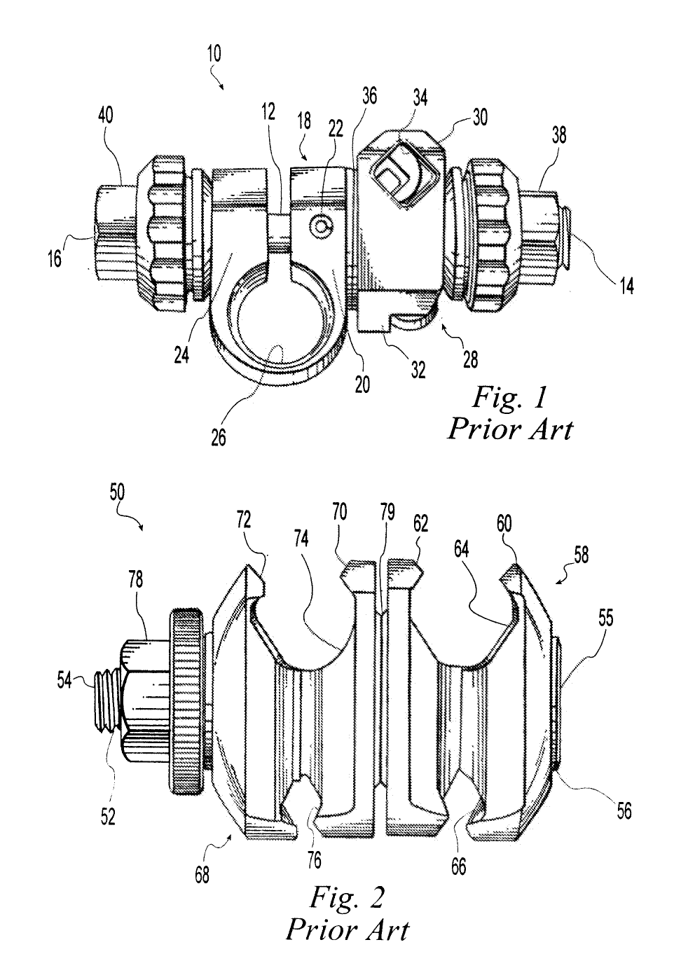

### DESCRIPTION OF THE ILLUSTRATIVE EXAMPLES

Embodiments of an orthopaedic fixation clamp according to the present invention include a plurality of clamp bodies mounted together to facilitate the interconnection of rods and bone fixation devices in an external fracture fixation device. Bone fixation devices may include bone pins, screws, wires, and/or other suitable bone fixation devices. For example, the bone fixation device may include a K-wire or Schanz screw. For simplicity, throughout this description, bone fixation devices are referred to generically as pins. The clamp may be configured and used to connect rods to one another, pins to one another, and rods to pins. The clamp may be used to assemble fixation constructs for performing fracture fixation of the long bones and pelvis, joint fusion, limb lengthening, osteotomies, fixation of periarticular fractures, and/or other orthopaedic procedures. The pins may be fixed in portions of the same bone as in separate fractured portions of a bone in a fracture fixation procedure. The pins may be fixed in portions of separate bones as in a joint immobilization procedure.

One or more of the clamp bodies may include openings for receiving both rods and pins such that the clamp body is able to alternatively and/or simultaneously grip both rods and pins such that the clamp is able to connect a rod to a rod, a rod to a pin, or a pin to a pin. For example, each clamp body may include a first opening sized to grip an interconnecting rod such as a carbon fiber reinforced composite rod, a metal rod, and/or other suitable rod. Each clamp body may include a second opening sized to grip a pin. The openings may open outwardly to permit the clamp to be connected to rods and/or pins without needing to slide them through the clamp. For example, the pin opening may be located inboard of the rod receiving opening and the pin opening may open into the rod opening and the rod opening may open outwardly such that a pin or rod may pass into the clamp body laterally over the side of the pin or rod. Each clamp body may include a plurality of pin and/or rod openings to permit more than one pin and/or rod to be connected to each clamp body.

The clamp may be configured to allow relative angular adjustment of the clamp bodies. For example, the clamp bodies may be mounted on a common shaft such that the bodies may be rotated relative to one another to accommodate various rod and/or pin constructs with various connection angles. The clamp bodies may be mounted to permit alternatively tightening one of the clamps while still permitting relative angular positioning of the clamp bodies. For example, the shaft may include one or more enlarged portions against which the plurality of clamp bodies abut and threaded ends receiving nuts to permit independent tightening of the clamp bodies. The enlarged portion and the clamp bodies may frictionally engage one another to prevent relative rotation when tightened or they may positively engage one another. For example, the enlarged portion and the clamp bodies may include interlocking serrations that allow the clamp bodies to be locked in a plurality of discrete angular positions relative to the shaft axis.

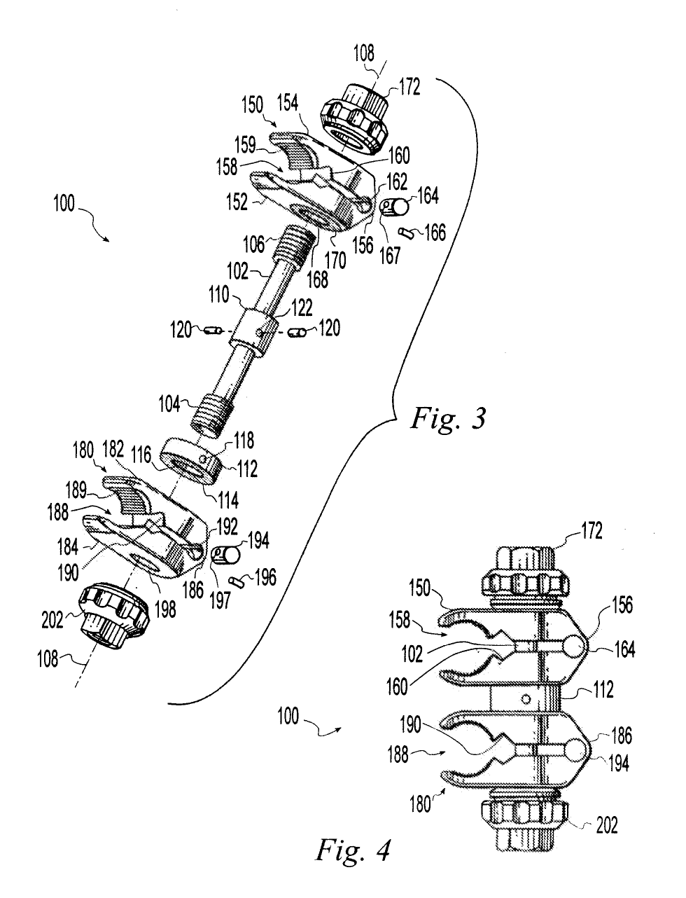

FIGS. 3-5 depict an exemplary orthopaedic fixation clamp 100. The clamp 100 includes a clamp shaft 102 and first and second clamp bodies 150, 180 mounted on the shaft for rotation and translation.

The shaft 102 includes a first threaded end 104, a second threaded end 106, and a shaft axis 108 extending therebetween. The shaft 102 includes an enlarged portion intermediate the first and second ends 104, 106 against which the clamp bodies 150, 180 abut to permit each clamp body to be independently rotated and tightened. In the exemplary embodiment, the shaft 102 includes an annular boss 110 extending radially outwardly intermediate the first and second ends 104, 106. The clamp bodies 150, 180 abut the boss 110 to permit each clamp body to be independently rotated and tightened.

Alternatively, a separate ring 112 may be mounted on the shaft 102 to provide an abutment surface. Providing a separate ring 112 facilitates forming serrations 114 on opposite faces (one shown) of the ring 112. The exemplary ring 112 includes a central axial bore 116 and a pair of opposing transverse bores 118 (one shown) communicating with the central bore 116. The ring 112 is mounted on the shaft 102 by engaging the central bore 116 with the boss 110 and then inserting pins 120 through the transverse bores 118 and seating the pins 120 in transverse bores 122 (one shown) in the boss 110.

The exemplary clamp bodies 150, 180 each include first 152, 182 and second 154, 184 legs connected together at a hinge line 156, 186. Each leg includes half of a rod receiving opening 158, 188 and half of a pin receiving opening 160, 190. The pin receiving openings 160, 190 open into the rod receiving openings 158, 188 and the rod receiving openings 158, 188 open outwardly to allow the clamp bodies 150, 180 to be positioned over a rod or pin by placing the clamp bodies 150, 180 laterally over the side of the rod or pin and without having to thread the rod or pin through the clamp body. The exemplary rod openings 158, 188 include optional serrations 159, 189 to improve their grip on a rod. The exemplary pin openings 160, 190 are smooth but they may include the optional serrations as well.

The exemplary clamp bodies 150, 180 are formed by machining the first 152, 182 and second legs 154, 184 from a solid block of material with a continuous interconnecting web of material forming a resilient hinge line 156, 186. The legs are separated from one another from the rod opening 158, 188 to the hinge line 156, 186. The unitary, hinged arrangement of the clamp bodies ensures that the openings in each leg will align with the openings in the opposite leg. A transverse bore 162, 192 is optionally formed at the hinge line 156, 186 to relieve stresses at the hinge line 156, 186 as it flexes when the clamps are tightened. A hinge pin 164, 194 is optionally positioned within the transverse bore 162, 192 to aid in tightening the clamp grip as the clamps are tightened. The hinge pin 164, 194 places the hinge line 156, 186 in tension when the clamps are tightened. The hinge pin 164, 194 is retained in the transverse bore 162, 192 by a pin 166, 196 inserted through the hinge line and into a bore 167, 197 in the hinge pin 164, 194.

The clamp bodies include transverse bores 168, 198 engageable with the shaft 102 and perpendicular to the rod and pin receiving openings. The clamp bodies 150, 180 are free to translate over the shaft until they abut the ring 112. The clamp bodies include annular serrations 170 (not shown on clamp body 180) formed on their inwardly directed faces so that they abut the serrations 114 formed on opposite sides of the ring 112 to permit each clamp body 150, 180 to be locked in a plurality of discrete angular positions relative to the ring 112. A nut 172, 202 threadably engages each threaded end 104, 106 of the shaft and may be independently tightened against the second leg 154, 184 of each clamp to compress the legs of each clamp together to grip a rod and/or pin and to lock each clamp body 150, 180 in a desired angular orientation relative to the ring 112.

In the exemplary clamp 100, the pin and rod openings are formed on one side of the clamp body opposite the hinge line with the shaft between the opening and the hinge line.

FIG. 4 shows the assembled clamp 100. The first clamp body 150 may be positioned over a rod or pin and tightened by tightening the first nut 172 to grip the rod or pin and lock the angular orientation of the clamp body 150 relative to the ring 112. By loosening the first nut 172, the position of the clamp along the rod or pin may be adjusted by sliding the clamp body 150 along the rod or pin and the angular orientation of the clamp body 150 relative to the ring 112 may be adjusted by rotating the serrations of the clamp body 150 over the serrations of the ring 112. Retightening the nut 172 locks the new positions. The second clamp body 180 may be adjusted in exactly the same way and totally independently of the first clamp body 150. The independence of the two clamp bodies, and their common universal design, permits gripping of pins and/or rods by either clamp body, and independent gripping and rotation of one clamp body relative to the other. For example, either of the clamp bodies may securely grip either a rod or pin while the other clamp body may be independently adjusted by repositioning it along another rod or pin and changing the angle of the clamp body relative to the ring 112 and consequently the other clamp body. For example, one clamp body may be securely tightened on a pin positioned within a first bone or bone fragment while the other clamp body remains loose to allow the other clamp body to be positioned and angled to set the first bone or bone fragment in a desired relationship to a rod.

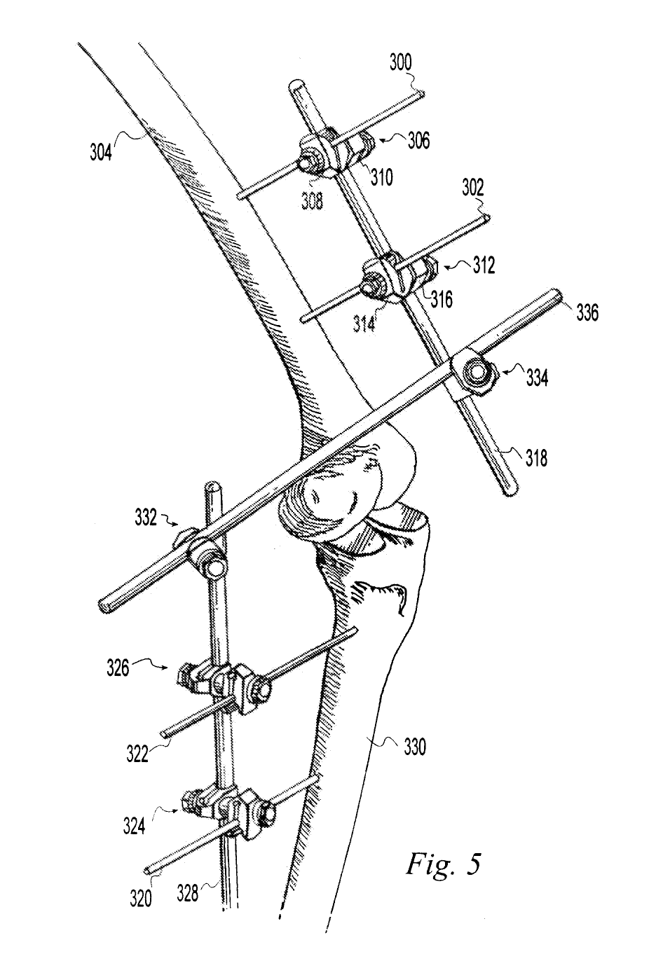

FIG. 5 illustrates an exemplary application of the clamp 100 in which a plurality of clamps is used to connect bone pins to rods to immobilize a knee joint. For example, first and second bone pins 300, 302 are inserted into the femur 304. A first clamp 306 is attached to the first pin 300 by tightening one of the clamp's 306 clamp bodies 308 securely to the pin 300. The other clamp body 310 is left loose for subsequent adjustment. A second clamp 312 is positioned with one of its clamp bodies 314 engaged with the second pin 302. Both clamp bodies 314, 316 of the second clamp remain loose to permit subsequent adjustment. A rod 318 is engaged with the clamp bodies 310, 316 of the first and second clamps 306, 312 that are not engaged with the pins 300, 302 and the rod and loose clamp bodies 310, 314, 316 are adjusted to position the rod 318 in a desired position relative to the pins 300, 302 and femur 304. The remaining clamp bodies 310, 314, 316 are tightened to lock the desired position. In the same way, bone pins 320, 322, third and fourth clamps 324, 326, and a rod 328 are attached to the tibia 330. The pin, clamp, and rod constructs on each of the femur 304 and tibia 330 form rigid connection points to fix the femur 304 and tibia 330 relative to one another. Two additional clamps 332, 334 and a rod 336 are used to connect the femoral and tibial rods 318, 328. The fifth clamp 332 is loosely engaged with the tibial rod 328 and the interconnecting rod 336. The sixth clamp 334 is loosely engage with the femoral rod 318 and the interconnecting rod 336. The tibia 330 and femur 304 are placed in a desired relative position and the fifth and sixth clamps 332, 334 are tightened to secure the bones in the desired position.

Although examples of an orthopaedic fixation clamp and its use have been described and illustrated in detail, it is to be understood that the same is intended by way of illustration and example only and is not to be taken by way of limitation. The invention has been illustrated in use to immobilize a knee joint. However, the fixation clamp may also be configured and used to accomplish other orthopaedic fixation and alignment tasks such as to perform immobilization of other skeletal joints, fracture fixation of the long bones and pelvis, joint fusion, limb lengthening, osteotomies, fixation of periarticular fractures, and/or other orthopaedic procedures. The clamp and constructs assembled using the clamp may be used as the primary means for accomplishing an orthopaedic procedure or as a temporary and/or supplemental way to support tissues and maintain alignment until another procedure such as periarticular plating or IM nailing can be performed. Accordingly, variations in and modifications to the orthopaedic fixation clamp and its use will be apparent to those of ordinary skill in the art, and the following claims are intended to cover all such modifications and equivalents.

What is claimed is:

1. An orthopaedic fixation clamp for gripping first and second elongated members, the clamp comprising:
an elongated clamp shaft having a first end, a second end, and a longitudinal shaft axis extending therebetween;
a first clamp body mounted to the clamp shaft for rotation about the shaft axis and having a first clamp opening able to receive the first elongated member;
a second clamp body mounted to the clamp shaft for rotation about the shaft axis and having a second clamp opening able to receive the second elongated member;
means for reducing the first clamp opening independently of the second clamp opening to securely grip the first elongated member;
means for locking the rotational position of the first clamp body relative to the shaft axis independently of the second clamp body;
means for reducing the second clamp opening independently of the first clamp opening to securely grip the second elongated member; and
means for locking the rotational position of the second clamp body relative to the shaft axis independently of the first clamp body.

2. The clamp of claim 1 wherein the means for reducing the first clamp opening comprises a first clamp body having first and second halves hinged together, the two halves being moveable toward one another along the shaft axis, the shaft having an enlarged portion intermediate the first and second ends, the first half of the first clamp body abutting the enlarged portion, and a nut threadably engaging the first end of the shaft and abutting the second half of the first clamp body such that advancing the nut along the shaft toward the first clamp body moves the second half toward the first half of the first clamp body to reduce the first clamp opening.

3. The clamp of claim 2 wherein the means for locking the rotational position of the first clamp body relative to the shaft comprises tightening the nut to press the first half of the first clamp body into engagement with the enlarged portion of the shaft.

4. The clamp of claim 3 wherein the first half of the first clamp body and the enlarged portion of the shaft each include serrations, the serrations of the first half being positively engageable with the serrations of the enlarged portion in a plurality of discrete angular positions of the first clamp body relative to the shaft.

5. The clamp of claim 4 wherein the enlarged portion of the shaft comprises a separate ring fixed to the shaft.

6. The clamp of claim 1 wherein the first clamp body includes a third clamp opening smaller than the first clamp opening such that the first clamp body may be alternatively clamped to elongated members having different diameters.

7. The clamp of claim 6 wherein the third clamp opening communicates with the first clamp opening and the first clamp opening opens outwardly such that the first clamp body is able to be placed over the side of the first elongated member to engage the first elongated member.

8. An orthopaedic fixation clamp assembly, comprising:
an elongated bone fixation member;
an elongated interconnecting rod; and
a clamp, the clamp comprising an elongated clamp shaft having a first end, a second end, and a longitudinal shaft axis extending therebetween; a plurality of clamp bodies, each body having a through bore engaged with the shaft for translation along the shaft axis, each body having two opposing halves defining a hinge between them on one side of the shaft axis, each body defining a relatively smaller fixation member receiving opening and a relatively larger rod receiving opening, each of the opposing halves defining one half of the fixation member receiving opening and one half of the rod receiving opening, the opposing halves being hingeable toward one another to reduce both openings and hingeable away from one another to enlarge both openings, both the fixation member receiving opening and the rod receiving opening being formed in each of the clamp bodies transverse to the shaft axis and opposite the hinge with the shaft between the openings and the hinge.

9. The assembly of claim 8 wherein the bone fixation member is selected from the group consisting of screws, pins, and wires.

10. The assembly of claim 8 wherein the two halves form a unitary body with a unitary, continuous, flexible hinge between them.

11. The assembly of claim 10 wherein the fixation member receiving opening is parallel to the rod receiving opening and wherein each clamp body includes a through bore parallel to the fixation member receiving and rod receiving openings, the through bore being positioned adjacent the hinge.

12. The assembly of claim 11 further comprising a hinge pin disposed within the through bore.

13. The assembly of claim 12 further comprising a pin extending through the hinge and into the hinge pin to secure the hinge pin within the through bore.

14. The assembly of claim 8 wherein the shaft includes an enlarged portion intermediate its first and second ends, the clamp bodies abutting opposing sides of the enlarged portion, the assembly further comprising a first nut threadably engaging the first end of the shaft and a second nut threadably engaging the second end of the shaft, the first and second nuts being threadably translatable along the shaft to independently press the first and second clamp bodies against the enlarged portion to press the opposing clamp body halves toward one another.

15. The assembly of claim 14 wherein each of the clamp bodies are further independently rotatable about the shaft axis relative to the enlarged portion.

16. The assembly of claim 15 wherein the enlarged portion of the shaft includes opposing outwardly directed axial faces, each face including serrations and each face abutting one of the clamp bodies, each of the clamp bodies including an inwardly directed axial face having serrations engageable with the serrations of the enlarged portion of the shaft to selectively lock each of the clamp bodies in one of a plurality of discrete angular positions independent of the other clamp body.

17. The assembly of claim 8 wherein the fixation member receiving opening is located inboard of the rod receiving opening, the fixation member receiving opening opening to the rod receiving opening and the rod receiving opening opening outwardly such that a fixation member or rod may pass into the clamp body laterally.

18. The assembly of claim 8 wherein each clamp body includes a through bore parallel to the fixation member receiving opening and the rod receiving opening, the through bore being positioned adjacent the hinge.

19. A method of fixing a first bone portion relative to a second bone portion, the method comprising:
inserting a first fixation member into the first bone portion and inserting a second fixation member into a second bone portion;
providing a first clamp having an elongated clamp shaft having a first end, a second end, and a longitudinal shaft axis extending therebetween; a first clamp body mounted to the clamp shaft for rotation about the shaft axis and having a first clamp opening able to receive an elongated member; a second clamp body mounted to the clamp shaft for rotation about the shaft axis and having a second clamp opening able to receive an elongated member;
engaging the first clamp opening with a portion of the first fixation member;
adjusting the first clamp body along the first fixation member;
adjusting the angle of the first clamp body relative to the shaft;
securing the first clamp body in fixed relation to the first fixation member;
locking the angle of the first clamp body relative to the shaft;
providing a second clamp having an elongated clamp shaft having a first end, a second end, and a longitudinal shaft axis extending therebetween; a third clamp body mounted to the clamp shaft for rotation about the shaft axis and having a third clamp opening able to receive an elongated member; a fourth clamp body mounted to the clamp shaft for rotation about the shaft axis and having a fourth clamp opening able to receive an elongated member;
engaging the third clamp opening with a portion of the second fixation member;
adjusting the third clamp body along the second fixation member;
adjusting the angle of the third clamp body relative to the shaft;
securing the third clamp body in fixed relation to the second fixation member;
locking the angle of the third clamp body relative to the shaft;
engaging a connecting rod with the second and fourth clamp openings;
adjusting the second and fourth clamp bodies along the connecting rod;
adjusting the angle of the second and fourth clamp bodies relative to their clamp shafts;
securing the second and fourth clamp bodies in fixed relation to the connecting rod; and
locking the angle of the second and fourth clamp bodies relative to their clamp shafts.
